(19) United States
(12) Patent Application Publication
(10) Pub. No.: US 2020/0107878 A1
(43) Pub. Date: Apr. 9, 2020

(54) Electrophysiology Device with Electrodes Having Increased Surface Area

(71) Applicant: St. Jude Medical, Cardiology Division, Inc., St. Paul, MN (US)

(72) Inventors: Gregory K. Olson, Elk River, MN (US); Andreas Ornberg, West St. Paul, MN (US); Edward E. Parsonage, St. Paul, MN (US); Chelsea Spencer, Minneapolis, MN (US)

(21) Appl. No.: 16/616,265

(22) PCT Filed: May 29, 2018
(86) PCT No.: PCT/US2018/034922

§ 371 Date: Nov. 22, 2019

Related U.S. Application Data
(60) Provisional application No. 62/516,740, filed on Jun. 8, 2017.

(51) Int. Cl. A61B 18/14

(52) U.S. Cl. CPC A61B 18/1492; A61B 2017/00526

(57) ABSTRACT

An electrophysiology catheter includes a catheter body and at least one porous electrode. The porous electrode is formed by forming a substrate of a first more noble metal and forming an alloy on the substrate. The alloy includes a second more noble metal and a less noble metal. The alloy is de-alloyed to form a porous matrix consisting essentially of the second more noble metal. The more noble metals can be gold, copper, and/or platinum, while the less noble metal can be silver, zinc, and/or lead.

### CROSS-REFERENCE TO RELATED APPLICATIONS

This application claims the benefit of U.S. provisional application No. 62/516,740, filed 8 Jun. 2017, which is hereby incorporated by reference as though fully set forth herein.

### BACKGROUND

The instant disclosure relates to catheters for use in medical procedures, such as electrophysiology studies. In particular, the instant disclosure relates to electrophysiology catheters that include electrodes with increased surface area and decreased impedance, both achieved by forming a porous surface on the electrodes.

Catheters are used for an ever-growing number of procedures, such as diagnostic, therapeutic, and ablative procedures, to name just a few examples. Typically, the catheter is manipulated through the patient's vasculature and to the intended site, for example, a site within the patient's heart.

A typical electrophysiology catheter includes an elongate shaft and one or more electrodes on the distal end of the shaft. The electrodes may be used for ablation, diagnosis, or the like. Oftentimes, these electrodes include ring electrodes that extend about the entire circumference of the catheter shaft, as well as a tip electrode.

As the dimensions of a measurement electrode decrease, the complex AC impedance with respect to a counter-electrode will generally increase. The increased impedance associated with smaller measurement electrodes can have undesirable effects during electrophysiology studies, such as electroanatomical mapping.

There are two primary contributing factors to the increased impedance. A first contributing factor is related to the dimensional dependence of the volumetric resistance (i.e., smaller electrodes have regions of higher current density). A second contributing factor is related to the capacitance of the electrode (i.e., as electrode dimensions shrink, the ionic AC current can become limited by how much charge can build up at the electrode surface). The second contributing factor, therefore, depends upon the total microscopic surface area of the electrode versus the macroscopic dimensional surface area of the electrode.

Various surface treatments are known to increase the surface area of a measurement electrode. Extant surface treatments, however, typically add material (e.g., iridium oxide; titanium nitride) to the measurement electrode, which complicates the manufacturing process.

### BRIEF SUMMARY

Disclosed herein is a method of manufacturing an electrophysiology catheter, including: forming a catheter body; forming a least one porous electrode according to a process including: forming a substrate of a first more noble metal; forming an alloy including a second more noble metal and a less noble metal on the substrate; and de-alloying the alloy to form a porous matrix consisting essentially of the second more noble metal; and securing the at least one porous electrode to the catheter body. The first more noble metal and the second more noble metal can be selected from the group consisting of gold, platinum, and copper; in embodiments, the first more noble metal is the same as the second more noble metal. The less noble metal can be selected from the group consisting of silver, zinc, and lead.

According to aspects of the disclosure, the alloy can be formed by co-depositing the second more noble metal and the less noble metal on the substrate via electrochemical plating. In other aspects of the disclosure, the alloy can be formed by co-depositing the second more noble metal and the less noble metal on the substrate via electroless plating. In still other aspects of the disclosure, the alloy can be formed by co-depositing the second more noble metal and the less noble metal on the substrate via physical vapor deposition. In further aspects of the disclosure, the alloy can be formed by co-depositing the second more noble metal and the less noble metal on the substrate via chemical vapor deposition. In yet further aspects of the disclosure, the alloy can be formed by: applying a layer of the less noble metal to a layer of the second more noble metal; and heating the layer of the less noble metal and the layer of the second more noble metal to allow inter-diffusion of the less noble metal and the second more noble metal.

De-alloying the alloy can include electrochemically dissolving the less noble metal from the alloy.

Also disclosed herein is an electrophysiology catheter formed according to a process including: forming a catheter body; forming a least one porous electrode according to a process including: forming a substrate of a first more noble metal; forming an alloy including a second more noble metal and a less noble metal on the substrate; and de-alloying the alloy to form a porous matrix consisting essentially of the second more noble metal; and securing the at least one porous electrode to the catheter body. The first more noble metal and the second more noble metal can be selected from the group consisting of gold, platinum, and copper, and can be either the same as each other or different from each other. The less noble metal can be selected from the group consisting of silver, zinc, and lead.

The alloy can be formed by co-depositing the second more noble metal and the less noble metal on the substrate. For example, the second more noble metal and the less noble metal can be co-deposited via electrochemical plating, via electroless plating, via physical vapor deposition, and/or by chemical vapor deposition.

Alternatively, the alloy can be formed by: applying a layer of the less noble metal to a layer of the second more noble metal; and heating the layer of the less noble metal and the layer of the second more noble metal to allow inter-diffusion of the less noble metal and the second more noble metal.

The alloy can be de-alloyed by electrochemically dissolving the less noble metal from the alloy.

The instant disclosure also provides an electrophysiology catheter including: a body; and at least one electrode disposed on the body, wherein the at least one electrode includes a substrate consisting essentially of a first noble metal and a porous matrix consisting essentially of a second noble metal on the substrate.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 schematically depicts an electrophysiology catheter and associated systems.

FIG. 2 is a close-up view of the distal region of the catheter shown in FIG. 1.

FIG. 3A schematically illustrates a porous electrode prior to a de-alloying process.

FIG. 3B schematically illustrates a porous electrode after a de-alloying process.

FIG. 4 is a microscopic photograph of a porous electrode according to aspects of the instant disclosure.

### DETAILED DESCRIPTION

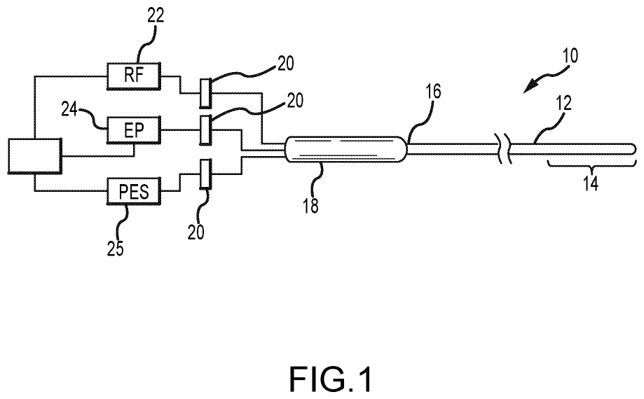

For purposes of illustration, the present teachings will be described in connection with a multi-electrode mapping and ablation catheter 10, such as illustrated in FIG. 1. As shown in FIG. 1, catheter 10 generally includes an elongate catheter body 12 having a distal region 14 and a proximal end 16. A handle 18 is shown coupled to proximal end 16. FIG. 1 also shows connectors 20. Connectors 20 are configured to be connected to a source of ablation energy (schematically illustrated as RF source 22, which can be, for example, the Ampere™ RF ablation generator of Abbott Laboratories), an electrophysiology mapping device (schematically illustrated as 24, which can be, for example, the EnSite Precision™ cardiac mapping system, also of Abbott Laboratories), and a programmable electrical stimulator (schematically illustrated as 25, which can be, for example the EP-4™ cardiac stimulator, also of Abbott Laboratories). Although FIG. 1 depicts three separate connectors 20, it is within the scope of the instant disclosure to have a combined connector 20 that is configured for connection to two or more of RF source 22, electrophysiology mapping device 24, and programmable electrical stimulator 25.

Various additional aspects of the construction of catheter 10 will be familiar to those of ordinary skill in the art. For example, the person of ordinary skill in the art will recognize that catheter 10 can be made steerable, for example by incorporating an actuator into handle 18 that is coupled to one or more steering wires that extend through elongate catheter body 12 and that terminate in one or more pull rings within distal region 14. Likewise, the ordinarily skilled artisan will appreciate that catheter 10 can be an irrigated catheter, such that it can also be coupled to a suitable supply of irrigation fluid and/or an irrigation pump. As a further example, those of ordinary skill in the art will appreciate that catheter 10 can be equipped with force feedback capabilities.

Insofar as such features are not necessary to an understanding of the instant disclosure, they are neither illustrated in the drawings nor explained in detail herein. By way of example only, however, catheter 10 can incorporate various aspects and features the following catheters, all from Abbott Laboratories: the EnSite™ Array™ catheter; the FlexAbility™ ablation catheter; the Safire™ BLU™ ablation catheter; the Therapy™ Cool Path™ irrigated ablation catheter; the Livewire™ TC ablation catheter; and the TactiCath™ Quartz irrigated ablation catheter.

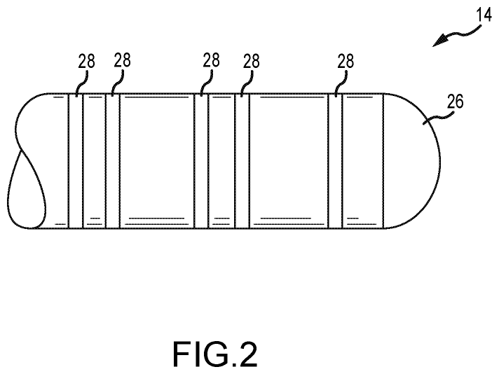

FIG. 2 is a close-up of distal region 14 of catheter 10. Distal region 14 of catheter 10 includes a tip electrode 26 positioned at its distal end and a plurality of additional electrodes 28 proximal of tip electrode 26. In particular, FIG. 2 depicts five ring electrodes 28. The person of ordinary skill in the art will understand and appreciate, however, that by varying the size (e.g., width) and spacing of electrodes 28, different diagnostic and/or therapeutic objectives and/or outcomes can be achieved. For example, the ordinarily skilled artisan will appreciate that, as electrodes 28 become smaller and closer together, the electrograms collected thereby will become sharper and more localized evidencing better depiction of local, near-field depolarization of the cardiac tissue in contact with the electrodes. Thus, it should be understood that distal region 14 can include any number of such electrodes 28 (e.g., 9 electrodes 28 for a decapolar catheter 10) and that the inter-electrode spacing can vary along the length of distal region 14.

Electrodes 28 may include any metal capable of detecting and conducting the local electrical signal. Suitable materials for electrodes 28 include, without limitation, platinum and gold.

Electrodes 28 can also be of various physical configurations. These include, by way of example only, ring electrodes, segmented ring electrodes, partial ring electrodes, flexible circuit electrodes, balloon electrodes, and spot electrodes. Various configurations of electrodes 28 (as well as electrode 26) are disclosed in International Publication No. WO 2016/182876, which is hereby incorporated by reference as though fully set forth herein.

The instant disclosure provides electrodes having a porous surface for increased microscopic surface area. More specifically, the instant disclosure provides noble metal electrodes including a matrix of pores that is formed through a de-alloying process (referred to herein as a “porous electrode”). Porous coatings formed by de-alloying processes will be familiar to those of ordinary skill in the art. See, e.g., Erlebacher et al., Evolution of Nanoporosity in Dealloying, Nature 410, 450-453 (Mar. 22, 2001), which is hereby incorporated by reference as though fully set forth herein. Thus, de-alloying processes will be described herein only to the extent necessary to understand the instant disclosure.

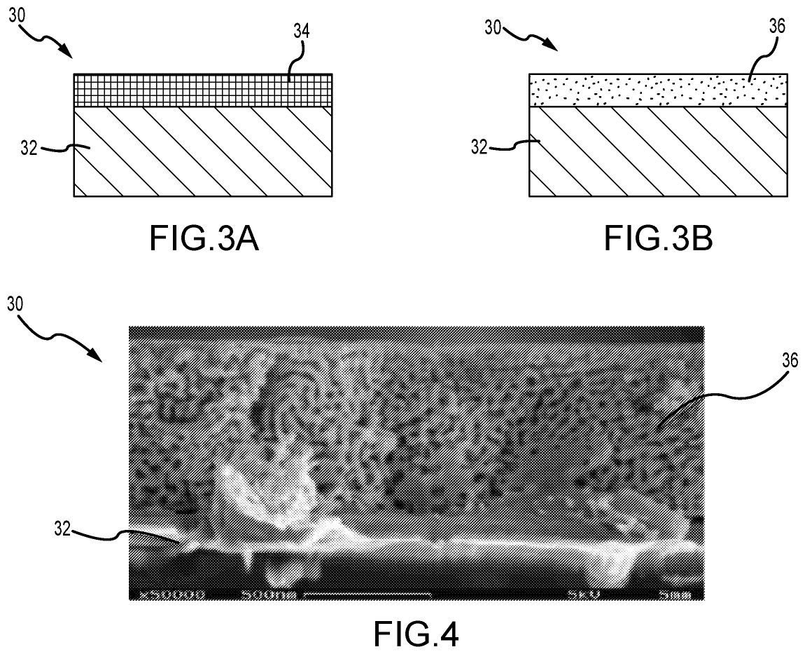

FIG. 3A schematically illustrates a porous electrode 30 prior to a de-alloying process. As shown in FIG. 3A, porous electrode 30 includes a substrate 32 and an alloy layer 34. Substrate 32 is made of a more noble metal, such as platinum, gold, or copper, while alloy layer 34 is an alloy of a more noble metal (which can be the same as or different from the more noble metal of substrate 32) and a less noble metal, such as silver, zinc, or lead.

In aspects of the disclosure, alloy layer 34 is formed by co-depositing the more- and less-noble metals, such as by electroless plating, electrochemical plating, physical vapor deposition, or chemical vapor deposition from a solution or atmosphere that contains the desired metal components. In other aspects of the disclosure, alloy layer 34 is formed by depositing a thin layer of the less noble metal on the surface of the more noble metal and then heating the resultant multi-layer structure to allow inter-diffusion of the metals.

In a de-alloying process, the less noble metal is removed from alloy layer 34, such as by electrochemical dissolution. For example, alloy layer 34 can be subjected to a voltage that will oxidize and solubilize the less noble metal, while leaving other alloy constituent metals (including the more noble metal) intact. This can be accomplished, for example, by using a 3-electrode configuration including alloy layer 34, a counter-electrode, and a reference electrode. It is also contemplated to add catalysts and/or other additives to the electrolyte solution to optimize the de-alloying process.

FIG. 3B schematically illustrates a porous electrode 30 after a de-alloying process. As shown in FIG. 3B, substrate 32 remains, and alloy layer 34 has been converted to a porous layer 36 including the more noble metal and a matrix of pores. The matrix of pores in porous layer 36 is visible in the microscopic photograph of FIG. 4.

It should be understood that the relative thicknesses of substrate 32 and alloy layer 34 are not to scale in FIG. 3A. Likewise, it should be understood that the relative thicknesses of substrate 32 and porous layer 36 are not to scale in FIG. 3B.

The matrix of pores in porous layer 36 can result in an increase in the microscopic surface area of porous layer 36 by about 20 times to about 40 times relative to a solid metal electrode. Another advantage of porous layer 36 according to the instant disclosure is an improvement in biosignal fidelity that results from a surface mechanical mismatch (as shown, for example, in connection with neuronal electrode development).

Although several embodiments have been described above with a certain degree of particularity, those skilled in the art could make numerous alterations to the disclosed embodiments without departing from the spirit or scope of this disclosure.

For example, the porous electrodes described herein can not only be formed prior to being attached to a catheter body, but can also be formed from non-porous electrodes already attached to a catheter body.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of manufacturing an electrophysiology catheter, comprising:
forming a catheter body;
forming a least one porous electrode according to a process comprising:
forming a substrate of a first more noble metal;
forming an alloy comprising a second more noble metal and a less noble metal on the substrate; and
de-alloying the alloy to form a porous matrix consisting essentially of the second more noble metal; and
securing the at least one porous electrode to the catheter body.

2. The method according to claim 1, wherein the first more noble metal and the second more noble metal are selected from the group consisting of gold, platinum, and copper.

3. The method according to claim 1, wherein the less noble metal is selected from the group consisting of silver, zinc, and lead.

4. The method according to claim 1, wherein forming the alloy comprises co-depositing the second more noble metal and the less noble metal on the substrate via electrochemical plating.

5. The method according to claim 1, wherein forming the alloy comprises co-depositing the second more noble metal and the less noble metal on the substrate via electroless plating.

6. The method according to claim 1, wherein forming the alloy comprises co-depositing the second more noble metal and the less noble metal on the substrate via physical vapor deposition.

7. The method according to claim 1, wherein forming the alloy comprises co-depositing the second more noble metal and the less noble metal on the substrate via chemical vapor deposition.

8. The method according to claim 1, wherein forming the alloy comprises:
applying a layer of the less noble metal to a layer of the second more noble metal; and
heating the layer of the less noble metal and the layer of the second more noble metal to allow inter-diffusion of the less noble metal and the second more noble metal.

9. The method according to claim 1, wherein de-alloying the alloy comprises electrochemically dissolving the less noble metal from the alloy.

10. The method according to claim 1, wherein the first more noble metal is the same as the second more noble metal.

11. An electrophysiology catheter formed according to a process comprising:
forming a catheter body;
forming a least one porous electrode according to a process comprising:
forming a substrate of a first more noble metal;
forming an alloy comprising a second more noble metal and a less noble metal on the substrate; and
de-alloying the alloy to form a porous matrix consisting essentially of the second more noble metal; and
securing the at least one porous electrode to the catheter body.

12. The electrophysiology catheter according to claim 11, wherein the first more noble metal and the second more noble metal are selected from the group consisting of gold, platinum, and copper.

13. The electrophysiology catheter according to claim 11, wherein the less noble metal is selected from the group consisting of silver, zinc, and lead.

14. The electrophysiology catheter according to claim 11, wherein forming the alloy comprises co-depositing the second more noble metal and the less noble metal on the substrate via electrochemical plating.

15. The electrophysiology catheter according to claim 11, wherein forming the alloy comprises co-depositing the second more noble metal and the less noble metal on the substrate via physical vapor deposition.

16. The electrophysiology catheter according to claim 11, wherein forming the alloy comprises co-depositing the second more noble metal and the less noble metal on the substrate via chemical vapor deposition.

17. The electrophysiology catheter according to claim 11, wherein forming the alloy comprises co-depositing the second more noble metal and the less noble metal on the substrate via electroless plating.

18. The electrophysiology catheter according to claim 11, wherein forming the alloy comprises:
applying a layer of the less noble metal to a layer of the second more noble metal; and
heating the layer of the less noble metal and the layer of the second more noble metal to allow inter-diffusion of the less noble metal and the second more noble metal.

19. The electrophysiology catheter according to claim 11, wherein de-alloying the alloy comprises electrochemically dissolving the less noble metal from the alloy.

20. An electrophysiology catheter comprising:
a body; and
at least one electrode disposed on the body, wherein the at least one electrode includes a substrate consisting essentially of a first noble metal and a porous matrix consisting essentially of a second noble metal on the substrate.
